(19) United States
(12) Patent Application Publication
(10) Pub. No.: US 2015/0105571 A1
(43) Pub. Date: Apr. 16, 2015

(54) METHOD FOR CONVERTING CARBON DIOXIDE AND BICARBONATES INTO FORMIC ACID DERIVATIVES USING A COBALT COMPLEX AS A CATALYTIC SYSTEM

(71) Applicant: Bayer Technology Services GmbH, Leverkusen (DE)

(72) Inventors: Ralf Jackstell, Rostock (DE); Carolin Ziebart, Rostock (DE); Christopher Federsel, Heidelberg (DE); Matthias Beller, Rostock (DE)

(21) Appl. No.: 14/361,146

(22) PCT Filed: Nov. 26, 2012
(86) PCT No.: PCT/EP2012/073550

§ 371 Date: May 28, 2014

(30) Foreign Application Priority Data
Nov. 29, 2011 (DE) 10 2011 087 302.3

(51) Int. Cl. B01J 31/24; C07C 51/00; C07C 231/14; C07C 51/41; C07C 67/08

(52) U.S. Cl. CPC B01J 31/2409; C07C 51/41; C07C 67/08; C07C 231/14; C07C 51/00; B01J 2231/625; B01J 2531/845; B01J 2231/62
USPC 556/7; 562/609; 560/265; 564/132; 556/18

(57) ABSTRACT

The invention relates to a method for converting carbon dioxide or bicarbonates into formic acid derivatives, i.e. formate salts, formate esters, and formamides, using molecular hydrogen and a catalytic system comprising a cobalt complex of cobalt salt and at least one tripodal, tetradentate ligand. The catalyst complex can be used as a homogeneous catalyst. The invention further relates to the cobalt complexes per se.

The invention relates to a process for reacting carbon dioxide or bicarbonates to produce formic acid derivatives using a catalytic system consisting of a cobalt complex formed from a cobalt salt and at least one tripodal tetradentate ligand. The catalyst complex can be used as a homogeneous catalyst. The invention also relates to the new cobalt complexes per se.

### PRIOR ART

The hydrogenation of carbon dioxide (scheme 1) and bicarbonates (scheme 2) is usually carried out using transition metal catalysts such as iridium, ruthenium and rhodium. For example, an iridium (III) catalyst generates 3 500 000 TON (turnover numbers) at 120° C. and 60 bar CO₂/H₂ after a reaction time of 48 hours (Nozaki (R. Tanaka, M. Yamashita, K. Nozaki, J. Am. Chem. Soc. 2009, 131, 14168-14169). Using a cationic Cp*Ir (III) catalyst having phenanthroline derivatives as ligands (Y. Himeda, N. Onozawa-Komatsuzaki, H. Sugihara, K. Kasuga, Organometallics 2007, 26, 702-712), a TON of 222,000 was achieved for the hydrogenation of carbon dioxide.

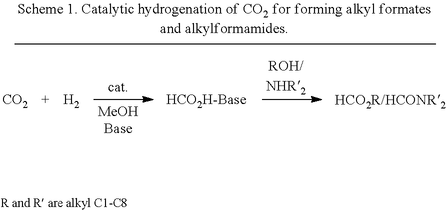

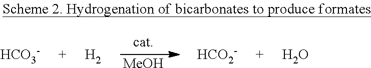

The highest yet TOF (turnover frequency), of about 95 000 h⁻¹, was achieved using a ruthenium catalyst (RuCl₂(OAc)(PMe₃)₄) and supercritical carbon dioxide (70 bar H₂/120 bar CO₂) (P. Munshi, A. D. Main, J. C. Linehan, C. C. Tai, P. G. Jessop, J. Am. Chem. Soc. 2002, 124, 7963-7971). For rhodium, the best result, with a TON of 3 400, was reported using various rhodium-phosphine complexes, such as RhCl(TPPTS)₃, at low pressure (F. Gassner, W. Leitner, J. Chem. Soc. Chem. Commun. 1993, 1465-1466).

For the hydrogenation of bicarbonates, the highest TON using only hydrogen was 2 500, wherein [RuCl₂(benzene)]₂ and dppm were used as catalyst (C. Federsel, R. Jackstell, A. Boddien, G. Laurenczy, M. Beller, ChemSusChem 2010, 3, 1048-1050).

Upon addition of hydrogen and carbon dioxide, a TON of 21 000 was achieved with an iridium catalyst (Y. Himeda, N. Onozawa-Komatsuzaki, H. Sugihara, H. Arakawa, K. Kasuga, Organometallics 2004, 23, 1480-1483).

Since the results for the hydrogenation of carbon monoxide and bicarbonates using base metal catalysts are too low, and additionally noble metal-containing catalysts have the disadvantage that they are costly, more cost-effective alternatives are sought. The Matthias Beller group reported the use of an Fe catalyst formed from [Fe(BF₄)₂].6H₂O and the tetradentate ligand P(CH₂CH₂PPh₂)₃ (T1), where a TON of 600 was achieved for the hydrogenation of carbon dioxide and bicarbonates. (C. Federsel, A. Boddien, R. Jackstell, R. Jennerhahn, P. J. Dyson, R. Scopelliti, G. Laurenczy, M. Beller, Angew. Chem. Int. Ed. 2010, 49, 9777-9780).

The use of a cobalt catalyst (cobalt film) which, however, operates only at high temperatures (250° C.) was described by U. Kestel, G. Fröhlich, D. Borgman, G. Wedler in Chem. Eng. Technol. 1994, 17, 390-396.

It is therefore the object of the invention to seek inexpensive catalyst systems of industrial utility for reacting carbon dioxide and bicarbonates, which catalyst systems achieve high activities and operate under simple reaction conditions, preferably at room temperature.

### DESCRIPTION OF THE INVENTION

The invention describes the use of cobalt complexes as catalysts for obtaining formic acid and formic acid derivatives at low temperatures (≦140° C.) and preferably at low pressure (≦100 bar) in good yield and at high conversions, using carbon dioxide or bicarbonates. The invention also relates to new cobalt complexes.

The process according to the invention is characterized in that a new catalyst system formed from a cobalt salt and at least one tripodal tetradentate ligand is used. The catalytic system can be used in the form of a homogeneous cobalt complex.

The catalyst can be separated off and reused after a reaction. The catalyst is stable over a wide temperature and pressure range.

When the described complex is used as a homogeneous catalyst complex, a suitable solvent should be used for carrying out the reaction. Suitable solvents for the reaction of carbon dioxide or bicarbonates to produce corresponding formic acid derivatives are selected from the group consisting of alcohols, for example methanol, ethanol, isopropanol, ethers, for example THF, dioxane, MTBE, ETBE, ketones, for example acetone, dibutyl ketone, amines, for example, monoethanolamine, amides, for example NMP, dimethylformamide, dibutylformamide, organic carbonates, for example propylene carbonate, and water.

The formic acid derivative (formate salt) formed can be any salt. The cation can be an organic cation or an inorganic cation, for example Li⁺, Na⁺, K⁺, Ca²⁺, Mg²⁺, Al³⁺, Fe²⁺, Co²⁺, Mn²⁺, NH₄⁺ or NEt₄⁺.

Bases, for example alkylamines (tert. or secondary), preferably tri- or diethylamine, are preferably added to the hydrogenation of carbon dioxide to produce corresponding formic acid derivatives such as formic acid-amine adducts, alkyl formates and/or formamides using the cobalt catalyst system according to the invention.

The reaction temperatures should generally be between 40° C. and 140° C. Preferably, the temperature range is from 60° C. to 120° C. Most preferably, the temperature range is from 80° C. to 120° C. Formic acid derivatives can be generated with high selectivity over the entire suggested temperature range. The hydrogen pressure should generally be between 5 and 100 bar.

The described catalyst system can consist of a catalyst, cobalt source and ligand generated in situ or it can consist of a previously synthesized cobalt complex.

Preference is given to using a catalyst system according to the invention which catalyst system is a cobalt complex consisting of a cation and an anion or consisting of a neutral cobalt complex having the general formula (1a) or (1b).

[Co(X)ₘ(L)ₙ]³⁰Y⁻ (Ia)

Co(X)ₘ(L)ₙ (Ib)
X, L, m and n are as follows:
X is selected from the group consisting of N₂, H₂, H, CO, CO₂, H₂O, halide, acetylacetonate (acac⁻), perchlorate (ClO₄²⁻), sulfate (SO₄²⁻) and formate (HCO₂⁻);
m is 1, 2, 3, 4, 5 or 6; preferably 1, 2 or 3;
n is 1 or 2.
L is a tripodal ligand of the general formula (II):

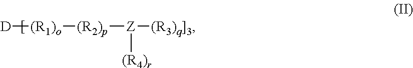

where
D and Z are the same or different and are selected from the group consisting of N, O, P and S;
o and p=0, 1, 2 or 3;
R₁ and R₂ are the same or different and are selected from the group consisting of alkyl (C1-C6), cycloalkyl (C3-C10) and aryl.
R₃ and R₄ are the same or different and are selected from the group consisting of alkyl (C1-C6), cycloalkyl (C3-C10), aryl or heteroaryl;
q and r=1 or 2;
where D and/or Z can be coordinated with the cobalt.
Y⁻is a monovalent anion selected from the group consisting of halides, P(R)₆⁻, S(R)₆³¹ , B(R)₄⁻, triflate and mesylate anions where R is an alkyl (C1-C6), cycloalkyl (C3-C6), aryl or halogen radical. Preferably, Y⁻=BF₄⁻, PF₆⁻or BPh₄⁻.

Halogen or halide comprises Cl, F, Br and I.

Examples of alkyl groups can include methyl, ethyl, n-propyl, isopropyl, n-butyl, sec-butyl, tert-butyl, n-pentyl and n-hexyl groups. As examples of cycloalkyl groups, cyclopropyl, cyclobutyl, cyclopentyl and cyclohexyl groups can be cited.

For the purposes of the invention, aryl denotes aromatic ring systems; these can be phenyl, naphthyl, phenanthryl, and anthracenyl.

Heteroaryl denotes heteroaromatic ring systems that may be five- or six-membered heterocycles, in which at least one carbon atom is replaced by nitrogen, oxygen and/or sulfur, preferably pyridine, quinoline, pyrimidine, quinazoline, furan, pyrazole, pyrrole, imidazole, oxazole, thiophene, thiazole or triazole.

m is preferably 1 or 2, n is preferably 1.

Preferable ligands of the general formula (II) are those in which D is nitrogen (N) or phosphorus (P). Z is preferably phosphorus (P).

R₁ and R₂ are the same or different and are preferably selected from the from the group alkyl (C1-C6) and/or phenyl. o and p are preferably 0 or 1, where at least o or p=1.

R₃ and R₄ are preferably phenyl. q and r are preferably 1.

Y⁻ is preferably BF₄⁻, PF₆⁻ or BPh₄⁻.

The invention also relates to the catalyst system of the general formula (1a) and (1b) per se where X, L, m and n are as defined above.

Preferably, the ligand to be used is a tetradentate ligand coordinated to the cobalt.

The most preferred ligands are
a) Tris(2-(diphenylphosphino)ethyl)phosphine [T1—(II) D and Z=P, R₁ and R₂=CH₂ and o and p=1 and also R₃ and R₄=phenyl and q=1 and r=1],
b) Tris(2-(diphenylphosphino)phenyl)phosphine, [T2—(II) D and Z=P, R₁=phenyl and o=1, p=0, and also R₃ and R₄=phenyl and q=1 and r=1].

If the complex is generated in situ, a cobalt source, as pre-catalyst, together with a ligand of the general formula (II) is used.

The cobalt source used can be a Co(0), Co(II) or Co(III) source. Preferred cobalt sources are Co(BF₄)₂.6H₂O, Co(acac)₂ or Co(acac)₃.

Particularly preferably used ligands are T1 or T2.

In this preferred embodiment (in situ catalyst) of the process according to the invention, an excess or a substoichiometric amount of the ligand is added to the cobalt source and, preferably, the cobalt source:ligand ratio is 1:1 or the ligand is present in excess.

Cobalt complexes of the general formula (1a), the use of which is very particularly preferred in the process according to the invention, are for example, [Co(acac)(T1)]BPh₄, [Co(acac)(T1)]BF₄, [CoH(T1)]BPh₄, [CoH(T1)]BF₄, [Co(H)₂(T1)]BPh₄, [Co(H)₂(T1)]BF₄ and [Co(H)₂(T1)]PF₆.

The preparation of cobalt catalyst complexes can be carried out as follows:

The synthesis of defined cobalt complexes was carried out according to the following literature reference: C. Bianchini, C. Mealli, A. Meli, M. Peruzzini, F. Zanobini, J. Am. Chem. Soc. 1988, 110, 8725-8726.

High activities (TON 3877) were achieved, for example, for the preparation of sodium formate from sodium carbonate, in various solvents and bases and at pressures of 5-80 bar using a preferably used in situ catalyst system formed from a Co source, preferably cationic Co(BF₄)₂.6H₂O, and the ligand tris[(2-diphenylphosphino)ethyl]phosphine (T1); MW 670.69052, melting point 134-139° C., such ligand being commercially available from Acros or Sigma Aldrich. Cobalt-catalyzed reactions generally cannot be employed in place of iron-catalyzed reactions and vice versa. Surprisingly, the catalyst activity was 6-fold higher than the best TON when one of the iron catalysts described in the literature is used, and 2-fold higher than when the best noble metal catalyst system is used.

The cobalt catalyst system preferably used in accordance with the invention is thereby superior even to previous systems that are based on the use of noble metal-containing catalyst systems.

### EXAMPLES

Ligand tris[(2-diphenylphosphino)ethyl]phosphine (T1); MW 670.69052, melting point 134-139° C., commercially available from Acros or Sigma Aldrich

### Example 1

Preparation of the Ligand T2

| Designation | Formula |
|---|---|
| T2 | 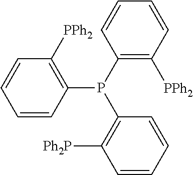 |

1 a) Preparation of T2 (Tris(2-(diphenylphosphino)phenyl)phosphine):

Under argon and with magnetic stirring, 1.5 g (4.4 mmol) of (2-bromophenyl)diphenylphosphine are dissolved in 30 ml of absolute THF (tetrahydrofuran) in a 100 ml three-necked flask equipped with a thermometer and a reflux condenser. The mixture is cooled to −78° C. using a cooling bath and at this temperature, 3 ml of 1.6 M n-butyllithium in hexane (4.8 mmol) is added to the mixture over 10 minutes using a dropping funnel. The mixture is stirred for 30 minutes at this temperature. Subsequently, at this temperature, 0.13 ml of phosphorus trichloride dissolved in 5 ml of absolute THF is added over 5 minutes. The reaction mixture is allowed to come to room temperature over one hour, with stirring, and is subsequently heated to reflux temperature (ca. 65° C.) for one hour. A cooling step is subsequently carried out and the solution is evaporated to dryness under reduced pressure. 30 ml of absolute toluene and 20 ml of (devolatilized) water are added. The toluene phase is washed three times with 20 ml of water and dried with magnesium sulfate. After filtration, evaporation to 10 ml under reduced pressure is carried out and 50 ml of absolute methanol is added to the solution. A white solid precipitates out over half an hour. This is the target product and it is filtered off and dried under reduced pressure. The yield is 0.6 g (50%) of tris(2-(diphenylphosphino)phenyl)phosphine. ¹H-NMR (300 MHz, CD₂Cl₂) δ (ppm): 6.5-7.3 m , ¹³C-NMR (75 MHz, CD₂Cl₂) δ (ppm): 128.4-128.8 (m), 129.0 (d, J_{PC}=21 Hz), 133.9-134.3 (m); 135.1-135.5(m) ³¹P-NMR (121 MHz, CD₂Cl₂) δ (ppm): −13.1-−14.5 (m, 3 P), −18.2- −23.5 (m, 1 P). HRMS: calculated for C₅₄H₄₂P₄: 814.22315; found: 814.221226.

### Example 2

Preparation of Cobalt Complexes

2a) Synthesis of [Co(H)T1]:

335 mg of T1 (0.5 mmol) was stirred, under argon, in 5 ml of absolute acetone. A black solution is formed by adding 129 mg (1 equivalent) of Co(acac)₂ and 5 ml of ethanol. After 10 minutes, 38 mg (2 equivalents) of NaBH₄ in 3 ml of ethanol is added. A yellow precipitate is formed. After 15 minutes, the steps of filtering, washing with ethanol and drying under reduced pressure are carried out. The ¹H NMR shows a quartet of doublets between δ −9.32 and −10.01 (J=42.2 Hz quart, 70.5 Hz d).

2b) General synthesis of [Co(H)₂T1]⁺ X⁻:

190 mg of the complex [Co(H)T1] is dissolved, under argon, in 10 ml of distilled THF. Subsequently, 1.5 equivalents of trifluoromethanesulfonic acid (0.035 ml) are added to the solution whereupon the color changes from orange to dark-red/black. After 10 minutes of stirring, the corresponding stoichiometric amount of the anion source is added as the sodium salt (NaBF₄, NaBPh₄, NH₄PF₆) in 5 ml of distilled ethanol. The solution is evaporated to half of its volume under reduced pressure. A filtering step is carried out and the solid is dried under reduced pressure. The catalyst can subsequently be used in hydrogenation experiments.

| Designation | General Formula |
|---|---|
| K1 | [Co(H₂)T1]BPh₄ |
| K2 | [Co(H)₂T1]PF₆ |
| K3 | Co(H)₂T1]BF₄ |
| K4 | [Co(H₂)T1]⁺BF₄⁻ |

2c) Preparation of K1, [Co(H₂)T1]⁺BPh₄⁻:

190 mg of the complex [Co(H)T1] is dissolved, under argon, in 10 ml of distilled THF. Subsequently, 1.5 equivalents of trifluoromethanesulfonic acid (0.035 ml) are added to the solution, whereupon the color changes from orange to dark-red/black. After 10 minutes of stirring, the corresponding stoichiometric amount of NaBPh₄, 15 mg, is added. The solution is evaporated to half of its volume under reduced pressure. A filtering step is carried out and the solid is dried under reduced pressure. The catalyst can subsequently be used in hydrogenation experiments. ¹H NMR: m, δ −10.84 −11.26.

2d) Preparation of K2, [Co(H)₂T1]⁺PF₆⁻:

189 mg of the complex [Co(H)T1] is dissolved, under argon, in 10 ml of distilled THF. Subsequently, 1.5 equivalents of trifluoromethanesulfonic acid (0.030 ml) are added to the solution, whereupon the color changes from orange to dark-red/black. After 10 minutes of stirring, the corresponding stoichiometric amount of NH₄PF₆, 63 mg, is added. The solution is evaporated to half of its volume under reduced pressure. 8 ml of ethanol is added. A filtering step is carried out and the solid is dried under reduced pressure. The catalyst can subsequently be used in hydrogenation experiments. ¹H NMR: m, δ −10.88-11.19.

2e) Preparation of K3, [Co(H₂)T1]⁺BF₄⁻:

190 mg of the complex [Co(H)T1] is dissolved, under argon, in 10 ml of distilled THF. Subsequently, 1.5 equivalents of trifluoromethanesulfonic acid (0.035 ml) are added to the solution, whereupon the color changes from orange to dark-red/black. After 10 minutes of stirring, the corresponding stoichiometric amount of NaBF₄, 43 mg, is added. The solution is evaporated to half of its volume under reduced pressure. 8 ml of ethanol is added. A filtering step is carried out and the solid is dried under reduced pressure. The catalyst can subsequently be used in hydrogenation experiments. ¹H NMR: m; δ −11.10 −11.36.

2f) Preparation of K4, [Co(H)₂T1]⁺BF₄⁻:

T1 (335 mg, 0.5 mmol) was stirred, under argon, in 5 ml of distilled acetone and 129 mg (1 equivalent) of Co(acac)₂ (dissolved in 5 ml of absolute ethanol) was added. A black solution resulted. After 10 minutes, 38 mg NaBH₄ (2 equivalents) and 3 ml of ethanol were added. A yellow/orange solid precipitates as a result. After 15 minutes of stirring, the yellow solid is filtered under argon and dried under reduced pressure.

The yellow solid is dissolved, under argon, in 10 ml of distilled THF and 0.035 ml of trifluoromethanesulfonic acid (1.5 equivalents) is slowly added to the solution whereupon the color changes from yellow to dark-red. After 10 minutes of stirring, 43 mg of NaBF₄ (1.5 equivalents) in 5 ml of distilled ethanol are added. The entire solution is evaporated to ca. 50% of its volume and the complex precipitates as a red solid. The steps of filtering and drying under reduced pressure step are carried out. ¹H NMR: m, δ_{H}=−11.10 −11.36.

### Example 3

Formation of sodium formate: 9.5 mg (0.028 mmol) of Co(BF₄)₂.6H₂O and 18.75 mg (1 equivalent) of T1 are dissolved in 40 ml of absolute methanol. 1.6 g of NaHCO₃ is placed in a 100 ml Parr autoclave and the preformed catalyst solution is introduced into the autoclave. The autoclave is flushed with hydrogen three times and pressurized with 60 bar of hydrogen at room temperature. The reaction mixture is subsequently heated to 80° C. and stirred for 20 hours at this temperature. Once the reaction time has elapsed, the autoclave is cooled down and the pressure is slowly released. The solution is evaporated to dryness at reduced pressure and the yield is calculated using ¹H NMR in D₂O, with THF as internal standard (relaxation time 20 seconds). All reactions were carried out in duplicate in order to guarantee reproducibility.

Various formic acid derivatives formed from carbon dioxide and sodium bicarbonate with in situ generation of the cobalt catalyst system using the cobalt source Co(BF₄)₂.6H₂O and the ligand tris[(2-diphenylphosphino)ethyl]phosphine (T1); MW 670.69052, melting point 134-139° C., this ligand being commercially available from Acros or Sigma Aldrich.

TABLE 1
Hydrogenation of sodium hydrogen carbonate

| | | | P_{H}_{₂}_{/CO}_{₂}^{[b]} | | | |
|---|---|---|---|---|---|---|
| No. ^{[a]} | L | Product | [bar] | T (° C.) | Yield(%)^{[c]} | TON |
| 1 | T1 | HCO₂Na | 60/0 | 80 | 94 | 645 |
| 2^{[d]} | T1 | HCO₂Na | 60/0 | 120 | 71 | 3877 |
| 3 | T1 | HCO₂Na | 5/0 | 100 | 23 | 155 |
| 4^{[e]} | T1 | HCO₂Na | 60/0 | 80 | 34 | 232 |
| 5[f] | — | HCO₂Na | 60/0 | 80 | 51 | 716 |
| 6[g] | T2 | HCO₂Na | 60/0 | 80 | 10 | 186 |

^{[a]} 9.5 mg (0.028 mmol) Co(BF₄)₂•6H₂O and 18.75 mg (1 eq) T1, 20 h, 20 ml MeOH, 3.3 g NaHCO₃
^{[b]}Pressure at room temperature
^{[c]}Sodium formate: yield using ¹H-NMR with THF as internal standard
^{[d]}amount of catalyst: 3.49 *10⁻⁶ mol
^{[e]}5 h
[f][Co(H₂)T1]⁺BF₄⁻
[g]Co(BF₄)₂•6H₂O 0.05 mol % (7.0 mg), T2 (17.0 mg), 40 ml MeOH, 39 mmol NaHCO₃

### Example 4

Formation of alkyl formates: The synthesis of the alkyl formates was carried out analogously to the synthesis of sodium formate, with the exception that 20 ml of the corresponding alcohol and 2 ml of triethylamine were added. The reaction mixture is subjected to 30 bar of CO₂ and 60 bar of hydrogen, in the autoclave, at room temperature, and subsequently the steps of heating to 100° C. and stirring for 20 hours are carried out. After cooling and releasing of the pressure, a GC analysis was carried out. This was carried out using a GC HP 6890N instrument with a 30 m HP5 column, internal diameter of 0.32 mm, 0.25 mm film, N₂ carrier gas, inlet temperature: 270° C., injection volume: 1 ml, split: 50:1, flow rate: 0.6 ml/min, after 20 minutes the flow rate was increased by 0.5 ml/min to 1 ml/min, T: 35° C. (until 20 minutes) then raised by 20° C. min⁻¹ up to 295° C. (hold for 17 minutes), detector temperature: 300° C., H₂ flow: 30 ml/min, air flow: 300 ml/min, makeup flow: 25 ml/min; with diglyme as internal standard. The yields are calculated as mol of product per mol of NEt₃.

TABLE 2
Hydrogenation of CO₂ to produce alkyl formates

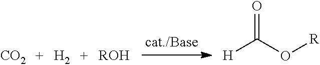

R = Alkyl

| | | | | | P H₂/ | | |
|---|---|---|---|---|---|---|---|
| | | mol of | Pro- | T | CO₂ | Yield | |
| No. | Cat./Ligand | NEt₃ | duct | (° C.) | (bar) | (%) | TON |
| 1 | Co(BF₄)₂/T1 | 0.014 | | 100 | 60/30 | 58 | 298 |
| 2 | Co(acac)₃/T1 | 0.014 | | 100 | 60/30 | 59 | 303 |
| 3 | [Co(NH₃)₆]Cl₃/T1 | 0.014 | | 100 | 60/30 | 83 | 427 |
| 4 | Co(II) chloride/T1 | 0.014 | | 100 | 60/30 | 69 | 355 |
| 5 | Co(BF₄)₂/T1 | 0.007 | | 100 | 60/30 | 85 | 219 |
| 6 | Co(BF₄)₂/T1 | 0.112 | | 100 | 60/30 | 16 | 659 |
| 7 | [Co(NH₃)₆]Cl₃/T1 | 0.112 | | 100 | 60/30 | 16 | 659 |
| 8 | [Co(H₂)T1]⁺ BPh₄⁻ | 0.014 | | 100 | 60/30 | 80 | 392 |
| 9 | Co(BF₄)₂/T1 | 0.007 | | 100 | 10/10 | 25 | 64 |
| 10 | Co(BF₄)₂/T1 | 0.007 | | 100 | 5/5 | 10 | 26 |
| 11 | Co(BF₄)₂/T1 | 0.014 | | 100 | 60/30 | 20 | 103 |
| 12 | [Co(H₂)T1]⁺BF₄⁻ | 0.014 | | 100 | 60/30 | 75 | 358 |

2.8 * 10⁻⁵ mol of catalyst and ligand and also 20 h reaction time in all reactions

### Example 5

Formation of formamides: The synthesis of the formamides was carried out analogously to the sodium formate synthesis, with the exception that 20 ml of methanol is replaced by 0.025 mol of the corresponding amine (dimethylamine, piperidine). The reaction solution is then pressurized with 30 bar CO₂ and 60 bar hydrogen and stirred for 20 hours at 100° C. The analysis was carried out using GC with diglyme as internal standard. The yields were calculated relative to the amine.

TABLE 3
Hydrogenation of CO₂ to produce formamides:

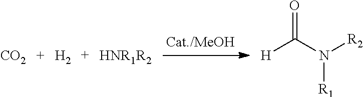

R₁, R₂ = Alkyl

| | | | | | P H₂/ | | |
|---|---|---|---|---|---|---|---|
| | | mol of | Pro- | T | CO₂ | Yield | |
| No. | Cat./Ligand | amine | duct | (° C.) | (bar) | (%) | TON |
| 1 | Co(BF₄)₂/T1 | 0.025 | | 100 | 60/30 | 77 | 687 |
| 2 | Co(BF₄)₂/T1 | 0.05 | | 100 | 60/30 | 73 | 1308 |
| 3 | Co(BF₄)₂/T2 | 0.04 | | 100 | 30/30 | 67 | 957 |
| 4 | Co(BF₄)₂/T1 | 0.025 | | 100 | 60/30 | 76 | 681 |
| 5 | Co(BF₄)₂/T1 | 0.05 | | 100 | 60/30 | 70 | 1254 |
| 6 | Co(BF₄)₂/T1 | 0.0125 | | 100 | 60/30 | 71 | 273 |

2.8 * 10⁻⁵ mol of catalyst and ligand and also 20 h reaction time in all reactions

1. A process for catalytically reacting carbon dioxide or bicarbonates to produce formic acid derivatives (formates), characterized in that a cobalt complex with at least one tripodal tetradentate ligand is used as catalyst system.

2. The process as claimed in claim 1, characterized in that the catalyst system is a cobalt complex consisting of a cation and an anion or consisting of a neutral complex having the general formula (1a) or (1b)
[Co(X)ₘ(L)ₙ]⁺Y⁻ (Ia)
Co(X)ₘ(L)ₙ (Ib)
where
X is selected from the group consisting of N₂, H₂, H, CO, CO₂, H₂O, halide, acetylacetonate (acac⁻), perchlorate (ClO₄²⁻) and sulfate (SO₄²⁻);
m is 1-6;
L is a tripodal ligand of the general formula (II) 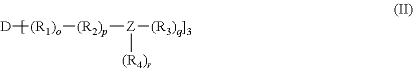
where
D and Z are the same or different and are selected from the group consisting of N, O, P and S;
o and p=0, 1, 2 or 3;
R₁ and R₂ are the same or different and are selected from the group consisting of alkyl (C1-C6), cycloalkyl (C3-C10) and aryl;
R₃ and R₄ are the same or different and are selected from the group consisting of alkyl (C1-C6), cycloalkyl (C3-C10), aryl and heteroaryl;
Q and r=1 or 2;
where D and/or Z can be coordinated with the cobalt;
n is 1 or 2; and
Y⁻ is a monovalent anion selected from the group consisting of halides, P(R)₆⁻, S(R)₆⁻, B(R)₄⁻, triflate and mesylate anions, where R is an alkyl (C1-C6), aryl or halogen radical.

3. The process as claimed in claim 1, characterized in that in the general formula (II), D is N or P.

4. The process as claimed in claim 1, characterized in that in the general formula (II), Z is P.

5. The process as claimed in claim 1, characterized in that the catalyst system has a ligand L of the general formula (II), which is selected from the following compounds:
a) tris(2-(diphenylphosphino)ethyl)phosphine [T1], and
b) tris(2-(diphenylphosphino)phenyl)phosphine, [T2].

6. The process as claimed in claim 1, characterized in that the catalyst system is used as a homogeneous complex in a solvent.

7. The process as claimed in claim 1, characterized in that the temperature is between 40 and 140° C.

8. The process as claimed in claim 1, characterized in that the catalyst system is generated in situ from a cobalt source and a tripodal ligand of the general formula (II), wherein a Co(O), Co(II), or Co(III) source is used.

9. The process as claimed in claim 1, characterized in that a cobalt complex selected from the group consisting of [Co(acac)(T1)]BPh₄, [Co(acac)(T1)BF₄, [CoH(T1)]BPh₄, [CoH(T1)]BF₄, [Co(H)₂(T1)]BPh₄, [Co(H)₂(T1)]BF₄ and [Co(H)₂(T1)]PF₆] is used as the catalyst system.

10. A catalyst system comprising a cobalt complex with a tripodal tetradentate ligand of the general formula (Ia) or (Ib)
[Co(X)ₘ(L)ₙ]⁺Y⁻ (Ia)
Co(X)ₘ(L)ₙ (Ib)
where L, X, Y, m and n are as defined in claim 2.

11. The catalyst system as claimed in claim 10, wherein the cobalt complex is selected from the group consisting of: [Co(acac)(T1)]BPh₄, [Co(acac)(T1)]BF₄, [CoH(T1)]BPh₄, [CoH(T1)]BF₄, [Co(H)₂(T1)]BPh₄, [Co(H)₂(T1)]BF₄, and [Co(H)₂(T1)]PF₆.

12. The process of claim 2, wherein m is 1, 2 or 3.

13. The process of claim 2, wherein Y⁻ is PF₆⁻, BF₄⁻ or BPh₄.
